(19) United States
(12) Patent Application Publication
(10) Pub. No.: US 2016/0183581 A1
(43) Pub. Date: Jun. 30, 2016

(54) POST-VAPORIZATION PLANT MATTER INFUSED SUBSTRATE FORMULATION

(71) Applicants: Stephen R. Martin, Quechee, VT (US); Stephen T.R. Martin, Quechee, VT (US)

(72) Inventors: Stephen T.R. Martin, Quechee, VT (US); Stephen R. Martin, Quechee, VT (US); Garrette David Victory Furo, Englewood, FL (US)

(73) Assignee: Stephen T.R. Martin, Quechee, VT (US)

(21) Appl. No.: 14/955,838

(22) Filed: Dec. 1, 2015

Related U.S. Application Data
(60) Provisional application No. 62/124,438, filed on Dec. 22, 2014.
(60) Provisional application No. 62/144,198, filed on Apr. 7, 2015.

(51) Int. Cl. A23D 7/005; A23C 15/00; A23D 9/00; A61K 36/185; C12G 3/00; C11B 9/00; A23L 1/226

(52) U.S. Cl. CPC A23L 1/3002; C12G 3/00; A23C 15/00; A23D 9/00; C11B 9/00; A61K 36/185; A23V 2002/00

(57) ABSTRACT

A process is provided for producing a substrate formulation comprising post-vaporization plant matter infused therein. The post-vaporization plant matter is gathered and saturated in a solvent to form a saturated mixture. The solvent is evaporated from the saturated mixture to form a reduced compound which is infused into a substrate thereby producing the post-vaporization plant matter infused substrate formulation.

### CROSS-REFERENCE TO RELATED APPLICATIONS

This application claims the benefit of each of the following U.S. Provisional Patent Applications, each of which is incorporated herein by reference in its entirety: (i) U.S. Provisional Patent Application Ser. No. 62/124,438 filed on Dec. 22, 2014; and (ii) U.S. Provisional Patent Application Ser. No. 62/144,198 filed on Apr. 7, 2015.

### TECHNICAL FIELD

The present invention is directed to providing a substrate formulation infused with a post-vaporization plant matter, and more particularly, infusing a substrate with plant matter that has been vaporized such as, for example, post-vaporized marijuana, lavender, tobacco and the like. The present invention is further directed to providing a commercially viable method for the collection of the vaporized plant matter.

### BACKGROUND

A vaporizer is a device that vaporizes the active ingredients of plant matter, such as, for example, cannabis, tobacco, or other herbs and oils or blends thereof for the purpose of inhalation. The vaporizer includes a heating source and a delivery system. Vaporizers typically include a lower chamber in which air is heated. The hot vapor rises through a filling chamber in which the plant matter is positioned, and the vapor and selected constituents of the plant matter are carried into a top chamber and captured within a vessel such as, for example, a food-grade plastic bag or balloon wherein the vapor begins to cool. Typically, a mouthpiece is coupled to the balloon and the vapor is inhaled.

Such a vaporization process eliminates the need to combust a selected plant matter and inhale the smoke produced from such combustion (i.e., “smoking”). Thus, because there is no combustion, no smoke is inhaled. In addition, the vaporized plant matter is not consumed by combustion (i.e., the plant matter is not burned via a flame). One embodiment of a known vaporizer is disclosed in U.S. Published Patent Application No. 2011/0308521 to Kofford; and commercial embodiments of a known vaporizer system are available from Storz & Bickel America Inc., Oakland, Calif., as a Volcano® Digit and a Volcano® Classic (Volcano® is a registered trademark of Stobi Gmbh & Co. KG Corporation of Germany).

In particular, smoking cannabis is known to provide certain positive benefits such as, for example, relief from nausea and pain. However, such positive benefits are overshadowed by associated health risks of smoking such as, for example, ingesting carcinogens and other toxins including ammonia, carbon monoxide and carbon dioxide.

While certain constituents of the plant matter placed into a filling chamber of a vaporizer are extracted, the amount or quantity of such plant matter is not diminished. It is commonplace for such vaporized plant matter, or post-vaporization plant matter, also known as “already-been-vaped” or “ABV” plant matter, to be discarded.

Vaporizers are known to be used as a means to inhale certain constituents of marijuana. There are approximately five hundred (500) identifiable chemical constituents known to exist in the cannabis plant, and at least eighty-five (85) different cannabinoids have been isolated from the plant. Cannabinoids are chemical compounds secreted by cannabis flowers that provide relief to an array of symptoms including pain, nausea and inflammation. Such cannabinoids imitate compounds that human bodies naturally produce, called endocannabinoids, the deficiency of which physically manifests itself in unbearable physical symptoms. One such cannabinoid is the phytocannabinoid tetrahydrocannabinol (“THC”), the primary psychoactive compound of cannabis. Other major constituents of the cannabis plant include cannabidiol (“CBD”) and cannabinol (“CBN”). Such constituents of the cannabis plant, THC, CBD and CBN, vaporize at a temperature near their respective boiling points: THC at about 157° C. (315° F.); CBD at about 170° C. (338° F.); and CBN at about 185° C. (365° F.). The use of vaporizers as a means to inhale marijuana is growing exponentially and, consequently, the generation of ABV marijuana is correspondingly growing exponentially.

Vaporizers are also known to be used as a means to inhale certain constituents of lavender. The main constituents of lavender include linalool, linalyl acetate, lavandulyl acetate, t-p3-ocimene, a-terpineol, nerol, neryl acetate and beta-caryophyllene. Linalool has been associated with stress reduction and thus lavender is vaporized for the inhalation of the linalool. Similarly, tobacco is vaporized for the inhalation of nicotine.

What is needed is an efficient, effective, economical process for conversion of the vaporized plant matter into a desirable product such as a food or beverage; as well as a means for providing a commercially viable method for the collection of the vaporized plant matter.

### SUMMARY

In one aspect, the present invention resides in a process for producing a substrate formulation comprising post-vaporization plant matter infused therein, the process comprising: a) gathering post-vaporization plant matter; and b) infusing the post-vaporization plant matter into a substrate; thereby producing the post-vaporization plant matter infused substrate formulation.

In another aspect, the present invention resides in a process for producing a substrate formulation comprising post-vaporization plant matter infused therein, the process comprising: a) gathering post-vaporization plant matter; b) exposing the post-vaporization plant matter to a solvent and saturating the post-vaporization plant matter in the solvent to form a saturated mixture; c) evaporating the solvent from the saturated mixture to form a reduced compound; and d) infusing the reduced compound into a substrate; thereby producing the post-vaporization plant matter infused substrate formulation.

### DETAILED DESCRIPTION

A vaporizer is a device that vaporizes the active ingredients of plant matter, such as, for example, cannabis, lavender, tobacco, or other herbs and oils or blends thereof (collectively referred to herein as “plant matter”) for the purpose of inhalation. A vaporizer typically includes: a heat source over which air passes as a result of inhalation by a user; a porous container in which the plant matter is disposed and through which the heated air passes; and an extraction chamber by which vaporized plant matter is passed to the user for subsequent inhalation.

In one embodiment, the vaporizer includes a heating source positioned within a lower chamber in which air is heated. The hot vapor rises through a filling chamber in which the plant matter is disposed. Typically, the filling chamber is sized to contain about one (1) gram to about five (5) grams of the plant matter. Preferably, such plant matter is finely ground before it is placed in the filling chamber. The vapor and selected constituents of the plant matter are carried into a delivery system for subsequent inhalation. In one embodiment, the vapor is passed into a top chamber of the vaporizer and is captured within a vessel such as, for example, a food-grade plastic bag or balloon wherein the vapor begins to cool. A mouthpiece is coupled to the balloon and the vapor is inhaled. The amount of plant matter that is placed into the filling chamber of the vaporizer may be subjected to multiple vaporization flows thereby filling the balloon multiple times. When such a vaporization flow no longer extracts the desired constituents of the plant matter, the vaporized plant matter, or already-been-vaped plant matter, is removed from the filling chamber.

The present invention provides an efficient, effective, economical process for the conversion or reclamation of post-vaporization plant matter, also referred to as “already-been-vaped” plant matter, and the infusion of such post-vaporization plant matter into a substrate to produce a substrate formulation. Such post-vaporization plant matter is referred to hereinafter as “ABV plant matter.” In one embodiment, the ABV plant matter is infused into an ingestible substrate, such as for example a food or beverage, to produce an ABV plant matter infused ingestible formulation (e.g., an alcoholic beverage, a butter, and the like). In other embodiments, the substrate formulation is an ABV plant infused perfume or an ABV plant infused oil. Substrates such as lavender, tobacco and marijuana have psychoactive compounds that can be reclaimed as ABV plant matter and such ABV plant matter can be incorporated into a variety of desired ABV plant infused substrate formulations. For example, THC and CBD can be harvested from ABV marijuana, linalool can be harvested from ABV lavender, and nicotine can harvested from ABV tobacco. In one embodiment, the present invention encompasses the process of infusing a food, beverage, perfume or oil, or the like with ABV plant matter. “ABV plant matter” as used herein after includes plant matter that has been subjected to heating, such as for example, heating via conduction, convection, radiation, or a combination of thereof, and further includes plant matter that has been subjected to pyrolisis.

In one embodiment, ABV plant matter is infused into an ingestible alcoholic beverage (i.e., the substrate) to produce an ABV plant matter infused alcoholic beverage (i.e., the substrate formulation), such as for example, an ABV tobacco infused whiskey. The ABV plant matter is gathered and placed into a first vessel. For example, one hundred (100) grams of ABV plant matter is gathered and placed into a glass beaker or other container such as, for example, a pot, pan, flask or mason jar. Preferably, the vessel is sized at the ratio of two thousand (2000) mL, volume per one hundred (100) grams of ABV plant matter. Subsequently, the ABV plant matter is exposed to an alcoholic beverage, such as for example, vodka, gin, whiskey, tequila, wine or the like, by mixing a sufficient amount of the alcoholic beverage with the ABV plant matter to at least soak and saturate the ABV plant matter. Preferably, the amount of the alcoholic beverage added to the first vessel exceeds the amount required to saturate the ABV plant matter by a factor of two or more. Preferably, the ABV plant matter infused alcoholic beverage stands for twenty four (24) hours. Thereafter, the ABV plant matter infused alcoholic beverage may be filtered to remove the plant matter prior to serving. Various filtering techniques are described herein below with respect to filtering of the ABV plant matter from the substrate formulation. Alternatively, the saturated ABV plant matter is filtered from the ABV plant matter infused alcoholic beverage to provide an alcoholic beverage-flavored plant matter, such as for example, for use in an ingestible product formulation or dried and subsequently smoked with or without other smokeable matter.

In one embodiment, ABV plant matter is infused into an ABV plant matter ingestible formulation that may subsequently be used in baking, cooking, or simply as a spread, such as for example, an ABV sage infused butter or cooking oil. The ABV plant matter is gathered and made available. For example, one hundred (100) grams of ABV plant matter is gathered. An ingestible butter is placed into a glass beaker or other heatable container. such as for example, a pot, pan or other glass jar. The butter is heated to a temperature such that the butter melts and the ABV plant matter is introduced into the melted butter and mixed therewith. Preferably, the temperature of the heated butter does not exceed a range of about 120° C. (about 250° F.) to about 135° C. (about 275° F.). The ABV plant matter infused butter may be filtered to remove the plant matter prior to serving. Various filtering techniques are described herein below with respect to filtering of the ABV plant matter from the substrate formulation. Thereafter, the ABV plant matter infused butter is cooled. Optionally, the ABV plant matter infused butter is cooled in a cooling vessel configured to mold a desired solid shape, such as for example, a conventional butter stick or other three-dimensional shape. While cooking or baking with ABV plant matter infused butter is described, the present invention is not limited in this regard as ABV plant matter may be directly used for baking and cooking without departing from the broader aspects of the present invention. For example, ABV sage may be used to bake sage infused cookies.

In one embodiment, the ABV plant matter is infused into a perfume, such as for example, an ABV lavender infused scented perfume. The named or generic plant matter typically incorporated into a formula or recipe selected for the creation of a particular perfume or scent is first subjected to vaporization and such vapor is consumed as desired. Subsequently, said ABV plant matter is gathered and made available for use in such formulation of said perfume. For example, a formulation for a lavender fragrance may require two grams of lavender. Instead, two (2) grams of ABV lavender is added to said formulation. Thus, an ABV lavender infused perfume is prepared.

In one embodiment, the ABV plant matter is infused into an oil, such as for example, an ABV infused lavender scented oil. The ABV plant matter is gathered and made available. For example, one hundred (100) grams of ABV plant matter is gathered. The oil is heated to a temperature such that the viscosity is lowered and the oil transitions into a more fluid state. The ABV plant matter is introduced into the oil and mixed therewith. Preferably, the temperature of the heated oil does not exceed a range of about 120° C. (about 250° F.) to about 135° C. (about 275° F.). The ABV plant matter infused oil may be filtered to remove the plant matter prior to serving. Various filtering techniques are described herein below with respect to filtering of the ABV plant matter from the substrate formulation. Thereafter, the ABV plant matter infused oil is cooled. For example, an ABV lavender infused scented oil may be prepared.

One embodiment of the present invention encompasses the process of infusing a product with the extract oil obtained or distilled from ABV plant matter. In one exemplary embodiment, an extract oil from ABV marijuana is obtained. The ABV marijuana may include cannabis stems, leaves, buds and other cannabis plant matter.

Initially, the ABV marijuana is substantially dried. For example, the saturated ABV marijuana is arranged or set out in a dry area or on a dry surface to aerate and fully dry. Preferably, the saturated ABV marijuana is set out to dry for at least six (6) hours. Such drying may be enhanced by a variety of drying or low-heat alternatives such as the use of a heat lamp. Any moisture absorbed into the ABV marijuana from the air will lower the final yield of the ABV marijuana. Alternatively, the ABV marijuana may be gathered and frozen until it is processed for the production of the extract oil.

The ABV marijuana is gathered and placed into a first vessel. For example, one hundred (100) grams of ABV marijuana is gathered and placed into a glass beaker or other container such as, for example, a pot, pan, flask or mason jar. Preferably, the vessel is sized at the ratio of two thousand (2000) mL volume per one hundred (100) grams of ABV.

Subsequently, the ABV marijuana is exposed to a solvent. The amount of solvent required to saturate and soak the ABV marijuana is a function of the size and shape of the first vessel; accordingly, there is no stoichiometric ratio of solvent-to-ABV marijuana. In one embodiment, the ABV marijuana is soaked or saturated in ethanol and/or cooked in fats. In one embodiment, the solvent is ethanol or isopropyl alcohol. In one embodiment, the solvent is diethyl ether. In some embodiments of the present invention, the solvent is acetonitrile (ACN), dimethylsulfoxide (DMSO), dimethylacetamide, dimethylformamide (DMF), tetrahydrofuran (THF), diethylether, dioxane, isobutyronitrile, benzonitrile, pyridine, diethylcarbonate, sulfolane, hexamethylphosphotriamide (HMPA/HMPT), n-butanol, sec-butanol, isobutanol, tert-butanol, chloroform, ethyl acetate, hexanes, pentane, pyridine, 1-butanol, 2-butanol, acetic acid, acetone, acetonitrile, benzene, t-butyl alcohol, methanol, or any combination thereof.

The solvent is poured into the first vessel such that the ABV marijuana contained therein is soaked in the solvent thereby producing a saturated mixture. The corresponding amount of solvent required to saturate one hundred (100) grams of ABV marijuana is in the range of about five hundred (500) mL to about one thousand (1000) mL. The saturated mixture is retained in the first vessel until the solvent evaporates. In one embodiment, the saturated mixture of the ABV marijuana and the solvent stands for approximately sixty (60) minutes at room temperature and the mixture preferably is stirred with a non-porous device such as, for example, a glass or metal rod. After the solvent is fully evaporated from the saturated mixture, a reduced compound is obtained.

In another embodiment, a heat source or heating element, such as for example a hot plate, is provided within a ventilated compartment, such as for example, within a. compartment having a fume hood arranged therein. The first vessel is placed on the hot plate in the fume hood. The fume hood is activated and the heat source is heated to a temperature that does not substantially exceed the boiling point of the solvent. For example, wherein the boiling point of ethanol is 78.4° C. (about 173° F.), the temperature of the heat source preferably does not exceed a temperature of about 85° C. (about 185° F.) to about 95° C. (about 203° F.); and wherein the boiling point of isopropyl alcohol is 82.6° C. (about 180° F.), the temperature of the heat source preferably does not exceed a temperature of about 90° C. (about 195° F.) to about 100° C. (about 212° F.). In the embodiment wherein the solvent is ethanol or isopropyl alcohol, it is preferable that the heating source is heated to a temperature in the range of about 65° C. (about 150° F.).

In one embodiment, the saturated mixture of the ABV marijuana and the solvent is exposed to such heat for a period of about twenty (20) minutes to about forty (40) minutes. Preferably, a magnetic stir bar operating at medium to high speed is placed within the first vessel throughout the heating operation. Alternatively, the saturated mixture is stirred with a non-porous device, such as for example, a glass or metal rod. The solvent substantially evaporates as a result of being exposed to the elevated temperature, the heat source is disengaged, and the reduced compound is obtained. Optionally, the solvent is entirely evaporated. In one embodiment wherein the solvent is ethanol and the substrate formulation is an ABV plant matter infused alcoholic beverage, the solvent is optionally not entirely evaporated. In one embodiment wherein the solvent is isopropyl alcohol, water is added to the reduced compound to cause further evaporation of any remaining solvent. Preferably, such additional water is in droplets.

In one embodiment, the reduced compound is passed into a filtering apparatus and into a second vessel thereby separating the ABV marijuana from a liquid extract. The formerly saturated ABV marijuana is captured in the filter and the oil extract or liquid extract is passed into the second vessel. Such filtering apparatus may include, but is not limited to, metal kitchen mesh or filtering cloth placed over an inlet of the second vessel. In one embodiment, the filtering apparatus includes a vacuum filtration system. The reduced compound is passed through a filter, such as for example a filter paper, positioned over the inlet of a separating device, such as for example a Buchner funnel, and a vacuum is activated and maintained until the saturated ABV ceases expelling the liquid extract. Optionally, the filtered ABV marijuana may be exposed to additional solvent and re-saturated thereby producing additional liquid extract. In one embodiment, the saturation and filtering of the ABV marijuana is repeated up to five times. Accordingly, the volume of the second vessel, or a total volume of a plurality of second vessels, should be up to five (5) times the volume of the first vessel.

Preferably, the second vessel(s) containing the yielded liquid extract is placed in a well-ventilated area, such as for example, under heat lamps next to a fan, under a fume hood, or outdoors such that any remaining solvent evaporates and an infusible product, or an extract oil, is obtained. Preferably, the solvent is recaptured, for example by the use of a still and stove. The extract oil may be infused within any substrate to produce an ABV plant infused substrate formulation.

In one embodiment, the second container(s) are placed under the fume hood and on the heat source. The fume hood is activated and the heat source is heated to a temperature in the range of about 30° C. (about 86° F.) to about 40° C. (about 104° F.); and preferably to a temperature of about 34.6° C. (about 95° F.). Any remaining solvent is evaporated and the extract oil is obtained therefrom.

While the above-described processes encompass obtaining or distilling an extract oil from ABV marijuana, the present invention is not limited in this regard as the above-described processes encompass obtaining or distilling an extract oil from other ABV plant matter or a combination of more than one type of ABV plant matter without departing from the broader aspects of the present invention. Extract oil may be obtained from other ABV plant matter according to the present invention for infusing a desired product, such as for example, mint for a menthol extract, St. Johns wort for a hyperforin abstract, and coffee grounds for a caffeine extract.

The ABV marijuana extract oil can be used in all of the formulations for products described herein above with respect to ABV plant matter. For example, ABV marijuana infused extract oil may be used in preparing: an ABV plant matter infused alcoholic beverage, such as for example, an ABV marijuana infused vodka; an ABV plant matter infused ingestible formulation, such as for example, an ABV marijuana infused butter or honey oil. In addition. ABV marijuana infused extract oil may be used in preparing varieties of plant based hashish, an inebriant concentrate made as a distillation of other inebriants.

In another embodiment, the present invention is directed to providing a commercially viable method for the collection of the vaporized plant matter. More particularly, the present invention provides a sustainable, renewable, customer-sourced base of low-cost heated plant matter for reuse and eventual resale, while also providing customers with the additional benefit of the free use of a vaporizer and reducing the unnecessary waste of useful plant matter.

In accordance with the present method, one or more vaporizers are provided. Preferably, the vaporizers are made available for use by patrons of a commercial establishment or business while patronizing said business. The plant matter to be vaporized is proffered by said business. Alternatively, similar to a bring-your-own-bottle (“BYOB”) commercial establishment or restaurant, said business optionally allows patrons to bring into said business their own plant matter to vaporize in the vaporizers provided by said business. Subsequent to vaporization of such plant matter, the ABV plant matter is collected by said business from such vaporizers or other viable sources. The collected ABV plant matter is subjected to one of the embodiments described herein above for the production of one or more ABV plant matter infused products.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A process for producing a substrate formulation comprising post-vaporization plant matter infused therein, the process comprising:
a) gathering post-vaporization plant matter; and
b) infusing the post-vaporization plant matter into a substrate;
thereby producing the post-vaporization plant matter infused substrate formulation.

2. The process of claim 1 wherein the substrate is an ingestible substrate and the substrate formulation is an ingestible formulation.

3. The process of claim 2 wherein the ingestible substrate is an alcoholic beverage and the ingestible formulation is an alcoholic beverage.

4. The process of claim 2 wherein the ingestible substrate is a butter and the ingestible formulation is a butter.

5. The process of claim 2 wherein the ingestible substrate is a cooking oil and the ingestible formulation is a cooking oil.

6. The process of claim 2 wherein the ingestible formulation is provided for at least one of baking food products and cooking food products.

7. The process of claim 1 wherein the substrate formulation is at least one of a scented perfume and a scented oil.

8. The process of claim 1 further comprising:
c) filtering the substrate formulation to remove remaining post-vaporization plant matter.

9. A process for producing a substrate formulation comprising post-vaporization plant matter infused therein, the process comprising:
a) gathering post-vaporization plant matter;
b) exposing the post-vaporization plant matter to a solvent and saturating the post-vaporization plant matter in the solvent to form a saturated mixture;
c) evaporating the solvent from the saturated mixture to form a reduced compound; and
d) infusing the reduced compound into a substrate;
thereby producing the post-vaporization plant matter infused substrate formulation.

10. The process of claim 9 further comprising:
drying the post-vaporization plant matter prior to exposing the post-vaporization plant matter to a solvent.

11. The process of claim 9 further comprising:
filtering the substrate formulation to remove remaining post-vaporization plant matter prior to infusing the reduced compound into a substrate.

12. The process of claim 9 wherein the solvent is at least one of ethanol, isopropyl alcohol, and diethyl ether.

13. The process of claim 9 wherein the step of evaporating the solvent from the saturated mixture to form a reduced compound includes heating the saturated mixture.

14. The process of claim 9 wherein the reduced compound is an extract oil.

15. The process of claim 9 wherein the post-vaporization plant matter is post-vaporization marijuana.
